(19) United States
(12) Patent Application Publication
(10) Pub. No.: US 2017/0151150 A1
(43) Pub. Date: Jun. 1, 2017

(54) ANTIPERSPIRANT COSMETIC COMPRISING SPECIFIC PROTEINS FROM HUMAN OR ANIMAL SOURCES OR SPECIFIC PROTEINS FROM FISH OR BIRDS OR EGGS, WHICH ARE EXEMPT OF ALUMINUM AND/OR ZIRCONIUM HALIDES AND/OR HYDROXY HALIDES

(71) Applicant: Henkel AG & Co. KGaA, Duesseldorf (DE)

(72) Inventors: Bernhard Banowski, Duesseldorf (DE); Stefan Evers, Haan (DE)

(73) Assignee: Henkel AG & Co. KGaA, Duesseldorf (DE)

(21) Appl. No.: 15/430,132

(22) Filed: Feb. 10, 2017

Related U.S. Application Data
(63) Continuation of application No. PCT/EP2015/064478, filed on Jun. 26, 2015.

(30) Foreign Application Priority Data
Aug. 26, 2014 (DE) 10 2014 216 895.3
Aug. 26, 2014 (DE) 10 2014 216 898.8
Aug. 26, 2014 (DE) 10 2014 216 901.1

(51) Int. Cl. A61K 8/64; B65D 25/02; A61K 8/19; B65D 85/00; A61Q 15/00; A61K 8/04

(52) U.S. Cl. CPC A61K 8/64; A61Q 15/00; A61K 8/046; A61K 8/19; B65D 85/70; B65D 25/02; A61K 2800/30; A61K 2800/88; A61K 2800/87; A61K 2800/524

(57) ABSTRACT

The invention relates to an antiperspirant cosmetic including at least one specific protein from human or animal sources or from fish or birds or eggs and which is exempt of aluminum and/or zirconium halides and/or hydroxy halides and Bovine serum albumin (BSA). The invention further relates to the use of a specific protein and to a non-therapeutic method for reducing body perspiration. Adding or using said at least one specific protein ensures that the sweat gland(s) is/are effectively influenced, thus resulting in a significant reduction in axillary hyperhidrosis even in the absence of antiperspirant aluminum salts.

### FIELD OF THE INVENTION

The present invention generally relates to an antiperspirant cosmetic agent that includes at least one substance, selected from the group of cosmetic oils that are liquid at 20° C. and 1013 hPa, odorants, and waxes, optionally at least one propellant, as well as special proteins from human or animal sources or special proteins from fish or birds or eggs. The cosmetic agent of the invention, however, includes neither aluminum and/or zirconium halides and/or hydroxyhalides nor bovine serum albumin (BSA). Adding at least one special protein results in the sweat gland(s) being influenced.

Furthermore, the present invention relates to a packaging unit (kit of parts) including a cosmetic agent of the invention and a cosmetic agent having at least one active antiperspirant substance.

Moreover, the present invention relates to the use of special proteins from human or animal sources or special proteins from fish or birds or eggs for at least partially influencing the sweat gland(s), the special protein not being bovine serum albumin (BSA).

In addition, the present invention relates to the use of a combination that includes at least one substance, selected from the group of cosmetic oils that are liquid at 20° C. and 1013 hPa, odorants, and waxes, optionally at least one propellant, as well as special proteins from human or animal sources or special proteins from fish or birds or eggs, for reducing and/or preventing sweat, in particular underarm sweat, or sweat in other body regions. The combination according to the invention includes neither aluminum and/or zirconium halides and/or hydroxyhalides nor bovine serum albumin (BSA).

Lastly, the present invention relates to a non-therapeutic cosmetic method for preventing and/or reducing body perspiration, in which an antiperspirant cosmetic agent of the invention or a packaging unit of the invention is applied to the skin, in particular to the skin of the armpits, and remains on the skin of the armpits for at least 1 hour, primarily for at least 2 hours, preferably for at least 4 hours, in particular for at least 6 hours.

### BACKGROUND OF THE INVENTION

Washing, cleansing, and hygiene of the body are a basic human need, and modern industry constantly attempts to meet these human needs in many ways. The constant elimination or at least reduction of body odor and underarm wetness is particularly important for daily hygiene. In the prior art, numerous specific deodorant or antiperspirant body care products are known which were developed for application in body regions with a high density of sweat glands, particularly in the armpit region. These are prepared in the most varied application forms, for example, as a powder, in the form of a stick, as an aerosol spray, pump spray, liquid and gel-like roll-on application, cream, gel, and as an impregnated flexible substrate (deodorant wipes).

Prior art cosmetic antiperspirants contain, apart from at least one oil or a wax and an odorant component or a perfume, at least one antiperspirant compound, in particular in the form of aluminum and/or zirconium halides and/or hydroxyhalides. These antiperspirant compounds, on the one hand, reduce body sweat secretion by a temporary narrowing and/or blocking of sweat gland excretory ducts, so that the amount of sweat can be reduced by about 20 to 60%. On the other hand, because of their antimicrobial action, they have an additional deodorizing effect.

Aluminum and/or zirconium halides and/or hydroxyhalides in conjunction with the acid pH of these antiperspirants can lead to unpleasant skin reactions in some users. Moreover, the use of the aforementioned antiperspirant compounds can lead to stain formation on clothing.

There is a need, therefore, to replace antiperspirant aluminum and/or zirconium halides and/or hydroxyhalides with other active antiperspirant cosmetic substances. These active antiperspirant substances should have good antiperspirant action, good skin tolerance, and easy formulatability. Moreover, these active antiperspirant substances should have no negative effect on the storage stability of the antiperspirant cosmetic agents.

The object of the present invention was to provide an antiperspirant cosmetic agent that avoids or at least reduces the disadvantages of the prior art and has good skin tolerance while simultaneously reliably reducing underarm wetness. Moreover, the antiperspirant cosmetic agent should have a high storage stability.

It was now found surprisingly that the use of at least one protein from human or animal sources or at least one protein from fish or birds or eggs in cosmetic agents results in an antiperspirant effect, which is virtually comparable to the antiperspirant action of formulations with aluminum salts and/or aluminum-zirconium complexes. The at least one protein in this case can cause a change in light absorption of 1 to 100% when the pH changes by at least 0.5 in a pH range of pH 4.0 to pH 8.0. The antiperspirant action of the agent of the invention is hereby achieved without the addition of antiperspirant aluminum and/or zirconium halides and/or hydroxyhalides and without addition of bovine serum albumin (BSA).

Furthermore, other desirable features and characteristics of the present invention will become apparent from the subsequent detailed description of the invention and the appended claims, taken in conjunction with this background of the invention.

### BRIEF SUMMARY OF THE INVENTION

An antiperspirant cosmetic agent, including at least one substance, selected from the group of cosmetic oils that are liquid at 20° C. and 1013 hPa, odorants, and waxes; propellant in a total amount of 0 to 99% by weight, based on the total weight of the antiperspirant cosmetic agent; and at least one protein in a total amount of 0.1 to 70% by weight, based on the total weight of the antiperspirant cosmetic agent, wherein the at least one protein is isolated from human or animal sources or from fish or birds or eggs and wherein the at least one protein causes a change in light absorption of 1 to 100% at a pH value change of at least 0.5 in a pH range of pH 4.0 to pH 8.0, a temperature of 20° C. to 40° C., and a protein concentration of 0.001 to 10% by weight, based on the total weight of the sample mixture used for determining the change in light absorption, wherein the antiperspirant cosmetic agent includes no aluminum and/or zirconium halides and/or hydroxyhalides and no bovine serum albumin (BSA).

A packaging unit (kit of parts), comprising, packaged separately from one another, at least one first container (C1), containing a cosmetic agent (M1) comprising at least one active antiperspirant substance; and at least one second container (C2), containing a cosmetic agent (M2) comprising at least one protein, wherein the at least one protein is isolated from human or animal sources or from fish or birds or eggs, wherein the at least one protein causes a change in light absorption of 1 to 100% at a pH value change of at least 0.5 in a pH range of pH 4.0 to pH 8.0, a temperature of 20° C. to 40° C., and a protein concentration of 0.001 to 10% by weight, based on the total weight of the sample mixture used for determining the change in light absorption, and wherein the cosmetic agent (M2) includes no aluminum and/or zirconium halides and/or hydroxyhalides and no bovine serum albumin (BSA).

Use of at least one protein for at least partially influencing the sweat gland(s), wherein the at least one protein is isolated from human or animal sources or from fish or birds or eggs, wherein the at least one protein causes a change in light absorption of 1 to 100% at a pH value change of at least 0.5 in a pH range of pH 4.0 to pH 8.0, a temperature of 20° C. to 40° C., and a protein concentration of 0.001 to 10% by weight, based on the total weight of the sample mixture used for determining the change in light absorption and wherein the at least one protein is not bovine serum albumin (BSA).

Use of a combination, including at least one substance, selected from the group of cosmetic oils that are liquid at 20° C. and 1013 hPa, odorants, and waxes; propellant in a total amount of 0 to 99% by weight, based on the total weight of the antiperspirant cosmetic agent; and at least one protein in a total amount of 0.1 to 70% by weight, based on the total weight of the antiperspirant cosmetic agent, wherein the at least one protein is isolated from human or animal sources or from fish or birds or eggs, wherein the at least one protein causes a change in light absorption of 1 to 100% at a pH value change of at least 0.5 in a pH range of pH 4.0 to pH 8.0, a temperature of 20° C. to 40° C., and a protein concentration of 0.001 to 10% by weight, based on the total weight of the sample mixture used for determining the change in light absorption, and wherein the combination includes no aluminum and/or zirconium halides and/or hydroxyhalides and no bovine serum albumin (BSA), for reducing and/or preventing sweat, in particular underarm sweat or sweat in other body regions.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The subject of the present invention is an antiperspirant cosmetic agent, including
a) at least one substance, selected from the group of cosmetic oils that are liquid at 20° C. and 1013 hPa, odorants, and waxes,
b) propellant in a total amount of 0 to 99% by weight, based on the total weight of the antiperspirant cosmetic agent, and
c) at least one protein in a total amount of 0.1 to 70% by weight, based on the total weight of the antiperspirant cosmetic agent, wherein the at least one protein is isolated from human or animal sources or from fish or birds or eggs and wherein the at least one protein causes a change in light absorption of 1 to 100% at a pH value change of at least 0.5 in a pH range of pH 4.0 to pH 8.0, a temperature of 20° C. to 40° C., and a protein concentration of 0.001 to 10% by weight, based on the total weight of the sample mixture used for determining the change in light absorption,
   wherein the antiperspirant cosmetic agent includes no aluminum and/or zirconium halides and/or hydroxyhalides and no bovine serum albumin (BSA).

A selective influence on the sweat gland(s) occurs by the use of the at least one protein from human or animal sources or from fish or birds or eggs with the aforementioned special physical properties in the antiperspirant cosmetic agents of the invention, without wishing to be restricted to this theory. Said selective influence on the sweat gland(s) can be, for example, a gel formation of the at least one protein at pH values that are exclusively within the excretory ducts of the sweat glands. An effective blocking of the excretory ducts of the sweat glands can be assured in this way, without the antiperspirant action of the cosmetic agent of the invention being reduced by premature unwanted gel formation due to the addition of the at least one special protein. Furthermore, the selective influence on the sweat gland(s) can also be a disturbance of the charge balance within the sweat gland(s), however, which leads to an influence on sweat production, in particular to a reduction of sweat production. Therefore, an effective reduction of underarm sweat is also assured in the absence of antiperspirant aluminum and/or zirconium halides and/or hydroxyhalides and in the absence of bovine serum albumin (BSA).

The term “antiperspirant” according to the invention is understood to mean the decrease or reduction of the perspiration of body sweat glands.

Moreover, the term “aluminum and/or zirconium halides and/or hydroxyhalides” in the context of the present invention is understood to mean in particular chlorides, bromides, and iodides of aluminum and zirconium and compounds of the formula Al(OH)_{y}X and Zr(OH)_{z}X, where X in the aforementioned formulas stands for a halide ion.

Furthermore, the term “cosmetic oil” in the context of the present invention is understood to mean an oil suitable for cosmetic use, which is not miscible with water in all amounts. The cosmetic oil used according to the invention is neither odorants nor essential oils.

Moreover, the term “odorants” in the context of the present invention is understood to mean substances with a molar mass of 74 to 300 g/mol, which include at least one osmophoric group in the molecule and have an odor and/or taste; i.e., they are capable of exciting receptors in the hair cells of the olfactory system. Osmophoric groups are groups bound covalently to the molecular skeleton in the form of hydroxy groups, formyl groups, oxo groups, alkoxycarbonyl groups, nitrile groups, nitro groups, azide groups, etc. In this regard, the term “odorants” in the context of the present invention also includes perfume oils that are liquid at 20° C. and 1013 hPa, perfumes, or perfume oil components.

Moreover, the term “waxes” in the context of the present invention is understood to mean substances that at 20° C. are kneadable or solid to hard and brittle, have a coarsely to finely crystalline structure, and in terms of color are translucent to opaque, but not vitreous. Furthermore, these substances melt above 25° C. without decomposition, are easily liquefiable (low viscous) slightly above the melting point, have a high temperature-dependent consistency and solubility, and can be polished under light pressure.

The term “proteins” according to the invention describes chemical compounds that form condensation products of amino acids, said products being linked acid amide-like by peptide bonds. The number of amino acids in the proteins is preferably 2 to 1000, preferably 2 to 500, in particular 2 to 60 amino acids. The term “proteins” according to invention is also understood to mean hydrolysates of a protein, which include protein fractions with different amino acid sequences and molecular weights. Moreover, this term in the context of the present invention is also understood to mean mixtures of proteins from human or animal sources or from fish or birds or eggs.

Furthermore, the term “proteins from animal sources” is understood to mean proteins that are isolated from animals that fall in the class of mammals.

Moreover, the term “change in light absorption of the at least one protein” is understood to mean both the positive and negative change in light transmittance of the sample mixture, in particular the protein solution, as well as the absorption of light by the at least one protein or sample mixture.

Furthermore, the term “pH change” is understood to mean the continuous change in the pH value. The continuous change in the pH value can be achieved, for example, by titration, or continuous addition, of a base or acid.

The term “sample mixture” according to the invention describes a mixture of the at least one special protein with a solvent, in particular water, buffer, or salt-containing aqueous solutions.

Moreover, the term “fatty acids,” as it is used in the context of the present invention, is understood to mean aliphatic carboxylic acids that have unbranched or branched carbon moieties having 4 to 40 carbon atoms. The fatty acids used in the context of the present invention can be both naturally occurring and synthetically produced fatty acids. Furthermore, the fatty acids can be mono- or polyunsaturated.

Lastly, the term “fatty alcohols” in the context of the present invention is understood to mean aliphatic, monohydric, primary alcohols, which have unbranched or branched hydrocarbon groups having 4 to 40 carbon atoms. The fatty alcohols used in the context of the invention can also be mono- or polyunsaturated.

The quantity given in percentage by weight in the present case, unless specified otherwise, refers to the total weight of the antiperspirant cosmetic agents of the invention.

The cosmetic agents of the invention include as the first component a) at least one substance, selected from the group of cosmetic oils that are liquid at 20° C. and 1013 hPa, odorants, and waxes.

In the context of the present invention, the cosmetic oil that is liquid at 20° C. and 1013 hPa is selected from the group of (i) volatile cyclic silicone oils, in particular cyclic and linear silicone oils; (ii) volatile nonsilicone oils, in particular liquid paraffin oils and isoparaffin oils; (iii) nonvolatile silicone oils; (iv) nonvolatile nonsilicone oils; and (v) mixtures thereof.

The term “volatile oil” according to the invention describes oils that at 20° C. and an ambient pressure of 1013 hPa have a vapor pressure of 2.66 Pa to 40,000 Pa (0.02 to 300 mm Hg), preferably of 10 to 12,000 Pa (0.1 to 90 mm Hg), more preferably of 13 to 3000 Pa (0.1 to 23 mm Hg), in particular of 15 to 500 Pa (0.1 to 4 mm Hg).

Moreover, the term “nonvolatile oils” in the context of the present invention is understood to mean oils that at 20° C. and an ambient pressure of 1013 hPa have a vapor pressure of less than 2.66 Pa (0.02 mm Hg).

It can be preferred according to the invention to use mixtures of volatile silicone oils and volatile nonsilicone oils in the antiperspirant cosmetic agents of the invention, because a drier skin feel is achieved as a result. Furthermore, in the context of the present invention it can be preferable, if the antiperspirant cosmetic agents include a nonvolatile silicone oil and/or a nonvolatile nonsilicone oil, so as to mask insoluble ingredients, such as talc or ingredients that dried on the skin.

Particularly preferred according to the invention is the use of mixtures of nonvolatile and volatile cosmetic oils, because parameters such as skin feel, visibility of residues, and stability of the antiperspirant cosmetic agent of the invention are established in this way and the agent can therefore be matched better to user requirements.

The volatile and nonvolatile silicone oils usable in the context of the present invention and volatile and nonvolatile nonsilicone oils are disclosed, for example, in the German unexamined patent applications DE 102010063250 A1 and DE 102012222692 A1.

According to a preferred embodiment of the present invention, the cosmetic oil that is liquid at 20° C. and 1013 hPa is included in a total amount of 0.02 to 98% by weight, preferably of 2 to 85% by weight, preferably of 4 to 75% by weight, more preferably of 6 to 70% by weight, even more preferably of 8 to 60% by weight, in particular of 8 to 20% by weight, based on the total weight of the antiperspirant cosmetic agent.

The cosmetic agent of the invention can also include at least one odorant as component a). Preferably, however, mixtures of different odorants are used, which together produce an attractive scent note. Odorants that can be used in the context of the present invention are, for example, disclosed in the German unexamined patent application DE 102010063250 A1.

Especially pleasingly scented antiperspirant cosmetic agents of the invention are obtained, when the at least one odorant is included in a total amount of 0.00001 to 15% by weight, primarily of 0.001 to 9% by weight, preferably of 0.01 to 8% by weight, more preferably of 0.1 to 7% by weight, even more preferably of 0.2 to 6% by weight, in particular of 0.2 to 2% by weight, based on the total weight of the antiperspirant cosmetic agent.

Furthermore, the antiperspirant cosmetic agents of the invention can include a wax as component a). Said wax is preferably selected from the group of (i) fatty acid glycerol mono-, di-, and triesters; (ii) Butyrospermum parkii (shea butter); (iii) esters of saturated monohydric C₈₋₁₈ alcohols with saturated C₁₂₋₁₈ monocarboxylic acids; (iv) linear primary C₁₂-C₂₄ alkanols; (v) esters of a saturated monohydric C₁₆₋₆₀ alkanol and a saturated C₈-C₃₆ monocarboxylic acid; (vi) glycerol triesters of saturated linear C₁₂₋₃₀ carboxylic acids, which may be hydroxylated; (vii) natural plant waxes; (viii) animal waxes; (ix) synthetic waxes; and (x) mixtures thereof. Waxes that can be used with preference in the context of the present invention are disclosed in the German unexamined patent application DE 102012222692 A1.

It is preferred, in the context of the present invention, if the wax is included in a total amount of 0.01 to 50% by weight, primarily of 3 to 40% by weight, preferably of 5 to 30% by weight, in particular of 6 to 25% by weight, based on the total weight of the antiperspirant cosmetic agent.

According to an embodiment of the present invention, it can be provided that the antiperspirant cosmetic agents of the invention include as component b) a propellant in a total amount of 0 to 99% by weight, based on the total weight of the antiperspirant cosmetic agent. If the cosmetic agent of the invention includes a propellant, said propellant is included preferably in a total amount of 1 to 98% by weight, primarily of 20 to 90% by weight, preferably of 30 to 85% by weight, in particular of 40 to 75% by weight, based on the total weight of the antiperspirant cosmetic agent. In this case, the cosmetic agents of the invention are produced as propellant gas-driven aerosols. Preferred propellants (propellant gases) are propane, propene, n-butane, isobutane, isobutene, n-pentane, pentene, isopentane, isopentene, methane, ethane, dimethyl ether, nitrogen, air, oxygen, laughing gas, 1,1,1,3-tetrafluoroethane, heptafluoro-n-propane, perfluoroethane, monochlorodifluoromethane, 1,1-difluoroethane, and tetrafluoropropenes, namely, both individually and also mixtures thereof. Hydrophilic propellant gases as well, such as, e.g., carbon dioxide, can be used advantageously in the context of the present invention, if the proportion of hydrophilic gases is selected as low and a lipophilic propellant gas (e.g., propane/butane) is present in excess. Propane, n-butane, isobutane, and mixtures of said propellant gases are particularly preferred. It emerged that the use of n-butane as the sole propellant gas can be particularly preferable according to the invention.

The antiperspirant cosmetic agent of the invention includes as third component c) at least one special protein from human or animal sources or from fish or birds or eggs.

An especially effective reduction of underarm sweat is achieved by the at least one special protein in the context of the present invention, if the at least one protein is included in a total amount of 0.5 to 60% by weight, primarily of 1.0 to 50% by weight, preferably of 1.5 to 40% by weight, more preferably of 2.0 to 30% by weight, in particular of 2.0 to 20% by weight, based on the total weight of the antiperspirant cosmetic agent. Without wishing to be restricted to this theory, the use of the aforementioned amounts of the at least one special protein results in a significant influence on the sweat gland(s) by gel formation of the protein in the excretory ducts of the sweat glands or by influencing the charge balance within the sweat gland(s). An excellent antiperspirant effect is assured in this way. Furthermore, the use of the aforementioned amounts of the at least one special protein does not lead to unstable formulations, so that the stability of the antiperspirant cosmetic agents of the invention is assured even over long storage periods.

Especially good results in regard to the decrease and/or reduction of underarm wetness and skin tolerance and storage stability are obtained, if the at least one protein has an average molecular weight M_{w} of 150 to 100,000 Da, primarily of 180 to 50,000 Da, preferably of 200 to 10,000 Da, more preferably of 250 to 8000 Da, in particular of 300 to 5000 Da. The average molecular weight M_{w} can be determined, for example, by gel permeation chromatography (GPC) (Andrews P.; “Estimation of the Molecular Weights of Proteins by Sephadex Gel filtration”; Biochem. J., 1964, 91, pp. 222 to 233).

According to a preferred embodiment of the present invention, the at least one protein has an isoelectric point that is in the range of pH 4.0 to pH 10.0, preferably of pH 4.0 to pH 9.5, in particular of pH 4.0 to pH 8.0. Proteins in particular that have an isoelectric point in the aforementioned pH range have proven to be advantageous in the context of the present invention with regard to the antiperspirant action and the stability of the cosmetic agents of the invention.

An especially high antiperspirant action, skin tolerance, and storage stability are assured in the context of the present invention, if the at least one protein causes a change in light absorption at a pH change of at least 0.5 in a pH range of pH 4.5 to pH 7.5, in particular of pH 5.0 to pH 7.0, at a concentration of 0.001 to 10% by weight of protein, based on the total weight of the sample mixture used for the pH measurement, and at a temperature of 20° C. Without wishing to be restricted to this theory, the use of the at least one special protein, which causes a change in light absorption in a specific pH range, results in a significantly increased influencing of the sweat gland(s) by pH-selective gel formation in the excretory ducts of the sweat glands or by a disturbance of the charge balance of the sweat gland(s). An excellent antiperspirant action of the cosmetic agents of the invention is assured in this way, which is comparable to the antiperspirant action of aluminum salt-containing or aluminum-zirconium salt-containing cosmetic agents of the prior art.

It is preferred in the context of the present invention, if the pH change is achieved by the addition of hydrogen carbonates or carbonates, in particular of sodium hydrogen carbonates.

According to a preferred embodiment of the present invention, the at least one protein is selected from the group of (i) unmodified proteins; (ii) hydrolyzed proteins; (iii) chemically modified proteins, in particular hydrophobically and/or cationically and/or anionically modified proteins; (iv) physically modified proteins, in particular fractionated and/or purified and/or irradiated proteins; (v) hydrolyzed unmodified proteins; (vi) hydrolyzed and chemically modified proteins, in particular hydrolyzed and hydrophobically and/or cationically and/or anionically modified proteins; (vii) hydrolyzed and physically modified proteins, in particular fractionated and/or purified and/or irradiated proteins; and (viii) mixtures thereof.

The term “unmodified proteins” according to the invention is understood to mean proteins that were treated neither by chemical methods such as, for example, hydrolysis or chemical modification, nor by physical methods such as, for example, purification, separation, or irradiation.

Furthermore, the term “hydrolyzed proteins” or “protein hydrolysates” is understood to mean proteins that are obtained by chemical, in particular alkaline or acid, hydrolysis, by enzymatic hydrolysis, and/or by a combination of the two types of hydrolysis. All enzymes with hydrolytic action, such as, for example, alkaline proteases, are suitable for the enzymatic degradation. Mixtures of individual amino acids, which are obtained only by mixing pure substances of the amino acids, and total hydrolysates, which consist only of individual amino acids, in the context of the present invention do not fall under the term “hydrolyzed proteins” or “protein hydrolysates.”

Moreover, the term “chemically modified proteins” in the context of the present invention is understood to mean proteins that are obtained by chemical reaction of the reactive groups of proteins, in particular the hydroxy, amine, imidazole, guanidino, and/or thiol groups of the side chains of the amino acids of the protein, with hydrophobic and/or cationic and/or anionic compounds.

In addition, the term “physically modified proteins” in the context of the present invention is understood to mean proteins that were modified by a physical effect, in particular by heat and/or light and/or fractionation.

In the context of said embodiment, it is particularly preferred if the at least one protein is selected from the group of chemically modified, in particular hydrophobically modified, proteins. In this regard, the hydrophobically modified protein has one or more C₄₋₃₀ carbon chains, wherein the C₄₋₃₀ hydrocarbon chains may be linear, cyclic, branched, unbranched, saturated, unsaturated, and aromatic and wherein the C₄₋₃₀ hydrocarbon chains are bound via ether and/or ester and/or amine and/or amide bonds to the protein moiety.

Moreover, it is preferred in the context of this embodiment, if the at least one protein is selected from the group of chemically modified, in particular cationically modified, proteins. Preferably, the cationically modified protein therefore includes one or more groups of the formula R¹—N⁺(CH₃)₂—CH₂—CH(OH)—CH₂—X—R, in which R¹ stands for an alkyl group having 1 to 30 carbon atoms, an alkenyl group having 1 to 30 carbon atoms, a hydroxyalkyl group having 1 to 30 carbon atoms, in particular for a methyl group, a C₁₀₋₁₄ alkyl group or a C₁₀₋₁₄ alkenyl group, X for O, N or S, and R for the protein moiety. The cationization of the proteins with the above-described groups can be achieved by reacting the proteins with suitable halides of the above formula, wherein the above-described groups can be bound to the protein via ether and/or ester and/or amide and/or amine bonds. The term “protein moiety” in the context of the present invention is to be understood to mean the backbone, formed by the linking of amino acids, of the appropriate protein to which the cationic group is bound via the aforementioned bonds.

According to another particularly preferred embodiment of the present invention, the at least one protein causes a change in light absorption of 1.5 to 90%, primarily of 2 to 80%, preferably of 2.5 to 70%, more preferably of 3 to 65%, and in particular of 3.5 to 60%. In particular, proteins from human or animal sources or from fish or birds or eggs, which cause the aforementioned change in light absorption, in the context of the present invention lead to an excellent antiperspirant action. The change in light absorption in this case can occur in response to a change in the light transmittance of the sample mixture, in particular due to turbidity, as well as the absorption of light by the sample mixture, in particular by the protein itself.

The changes in light absorption, underlying this invention, at a change in pH of at least 0.5 can be determined by measuring the light transmission of a light beam through the sample mixture. The light transmission is measured using a Metrohm Optrode 6.1115.000 at a wavelength of 574 nm (greenish yellow) in mV (resolution of 0.1 mV) in an open sample container at 23° C. and 1013 mbar. The pH change in the pH range of 4.0 to 8.0 is achieved by the slow and continuous addition of a carbonate or hydrogen carbonate solution, preferably a 1% by weight sodium hydrogen carbonate solution, to the sample mixture during measurement of the pH using a pH electrode and with stirring at a speed of 750 to 850 rpm. The change in light absorption, caused by the at least one protein, is calculated according to the formula ΔL=[(|Lᵢ|/|L₀|]*100. In this formula, Lᵢ stands for the light transmission before and after the change in pH by at least 0.5 in the pH range of 4.0 to 8.0, preferably of pH 4.5 and 7.5, in particular of pH 5.0 and 7.0, therefore, for example, light transmission at pH 5.0 minus the light transmission at pH 6.0. L₀ in this formula stands for the difference of the light transmission at pH 4.0 and pH 8.0, preferably at pH 4.5 and pH 7.5, in particular at pH 5.0 and pH 7.0, therefore, for example, the light transmission at pH 8.0 minus the light transmission at pH 4.0. The at least one special protein in the antiperspirant cosmetic agents of the invention causes a change in light absorption of 1 to 100%, as determined by the above method. The present invention, however, is not limited to antiperspirant cosmetic compositions that contain at least one special protein, which causes a change in light absorption of 1 to 100%, as determined by the above method. It also comprises antiperspirant cosmetic compositions that contain at least one special protein, which causes a change in light absorption of 1 to 100%, as determined by other methods.

It is preferred in the context of the present invention, if the concentration of the at least one protein in the mixture, used for determining the change in light absorption, is 0.005 to 10% by weight, primarily of 0.05 to 5% by weight, preferably of 0.07 to 3% by weight, in particular of 0.09 to 2% by weight, based on the total weight of the sample mixture used for determining the change in light absorption.

According to the invention, preferably the at least one protein causes a change in light absorption at a pH change of at least 0.5 and at most 3.5, preferably of at least 0.5 and at most 2.5, in particular of at least 0.5 and at most 1.5. The change in the pH value can be brought about in particular by adding acids or bases, preferably bases in the form of carbonates or hydrogen carbonates, in the appropriate amount.

According to another preferred embodiment of the present invention, the antiperspirant cosmetic agent has a pH value of pH 2 to pH 10. A stable formulation of the cosmetic agents of the invention is possible within this range, without unwanted interactions occurring between the ingredients of the antiperspirant cosmetic agents of the invention. The desired pH can be established according to the invention by the use of acids and bases known to the skilled artisan and typical in antiperspirant cosmetic agents.

It is preferred, furthermore, according to the invention, if the antiperspirant cosmetic agent includes in addition at least one preservative. Preservatives preferred according to the invention are the formaldehyde releasers, iodopropynyl butylcarbamates, parabens, phenoxyethanol, ethanol, benzoic acid and salts thereof, dibromodicyanobutane, 2-bromo-2-nitropropane-1,3-diol, imidazolidinyl urea, 5-chloro-2-methyl-4-isothiazolin-3-one, 2-chloracetamide, benzalkonium chloride, benzyl alcohol, salicylic acid, and salicylates. Further, preservatives that may be used in the context of the present invention are the substances listed in Annex 6 of the German Cosmetics Act and cosmetic raw materials with preserving properties or raw materials that support or enhance the preserving action of the aforementioned preservatives. The preservatives are preferably included in a total amount of 0.01 to 10% by weight, primarily of 0.1 to 7% by weight, preferably of 0.2 to 5% by weight, in particular of 0.3 to 2.0% by weight, based on the total weight of the antiperspirant cosmetic agent.

It is preferred in the context of the present invention, if the antiperspirant cosmetic agent is present as a water-in-oil emulsion. In this case, this can be in particular a sprayable water-in-oil emulsion, which can be sprayed using a propellant. It is preferred in this regard, if the antiperspirant cosmetic agent of the invention, which has the form of a water-in-oil emulsion, includes the at least one protein in a total amount of 0.1 to 70% by weight, primarily of 0.5 to 60% by weight, preferably of 1.0 to 50% by weight, more preferably of 1.5 to 40% by weight, even more preferably of 2.0 to 30% by weight, in particular of 2.0 to 20% by weight, based on the total weight of the antiperspirant cosmetic agent.

It can be equally preferred according to the invention, however, if the antiperspirant cosmetic agent is present as an oil-in-water emulsion. In this case, the cosmetic agent of the invention is preferably sprayed as a propellant-free pump spray or squeeze spray or applied as a roll-on. It is preferred in this regard, if the antiperspirant cosmetic agent, which has the form of an oil-in-water emulsion, includes the at least one protein in a total amount of 0.1 to 70% by weight, primarily of 0.5 to 60% by weight, preferably of 1.0 to 50% by weight, more preferably of 1.5 to 40% by weight, even more preferably of 2.0 to 30% by weight, in particular of 2.0 to 20% by weight, based on the total weight of the antiperspirant cosmetic agent.

According to another preferred embodiment of the present invention, the cosmetic agents of the invention may contain only a small amount of free water or no free water. Free water in the context of the present invention is understood to be water that is different from water of crystallization, water of hydration, or similar molecularly bound water of the employed components. The antiperspirant cosmetic agent preferably includes free water in a total amount of less than 10% by weight, primarily of less than 8% by weight, preferably of less than 5% by weight, more preferably of less than 3% by weight, even more preferably of less than 1% by weight, in particular of 0% by weight, based on the total weight of the antiperspirant cosmetic agent.

It is also preferred according to the invention in the context of a further embodiment, however, if the antiperspirant cosmetic agent is present as an aqueous, aqueous-alcoholic, or aqueous-glycolic solution. Because the cosmetic agents of the invention contain no antiperspirant aluminum and/or zirconium halides and/or hydroxyhalides, which have a reduced antiperspirant action due to the addition of protic solvents, according to the invention protic solvents, such as aqueous solutions, can be used for formulating the cosmetic agents of the invention, without a significant reduction in antiperspirant action. Therefore, the addition of the at least one special protein itself when protic solvents are used assures an effective influence on the sweat gland(s) and thereby an excellent antiperspirant action.

In regard to this embodiment of the present invention, it was found surprisingly that the influence on the sweat gland(s) by the at least one special protein can be significantly increased, if the antiperspirant cosmetic agents of the invention contain free water in an amount of 5 to 99% by weight, based on the total weight of the antiperspirant cosmetic agent. In a particularly preferred embodiment of the present invention, the antiperspirant cosmetic agent therefore includes free water in a total amount of 5 to 96% by weight, primarily of 15 to 80% by weight, preferably of 30 to 70% by weight, in particular of 40 to 60% by weight, based on the total weight of the antiperspirant cosmetic agent.

Furthermore, it is preferred in regard to this embodiment, if the antiperspirant cosmetic agent includes ethanol in a total amount of 1 to 99% by weight, primarily of 5 to 70% by weight, preferably of 7 to 50% by weight, in particular of 10 to 30% by weight, based on the total weight of the antiperspirant cosmetic agent. As previously stated, high amounts of protic solvents, such as ethanol, can be used with the use of the at least one special protein itself, without the antiperspirant action of the antiperspirant cosmetic agent of the invention being negatively influenced.

The antiperspirant cosmetic agent of the invention can be applied by different methods. According to a preferred embodiment, the antiperspirant cosmetic agent is produced as a spray application. The spray application occurs with a spray device, which contains in a container a filling of the liquid, viscous-flowable, suspension-like, or powdered antiperspirant cosmetic agent of the invention. The filling can be under the pressure of a propellant (compressed-gas cans, compressed-gas dispensers, aerosol dispensers), or this can refer to a pump sprayer, to be operated mechanically, without a propellant gas (pump spray/squeeze bottle). The spraying of the antiperspirant cosmetic agent can occur in this case physically, mechanically, or electromechanically, for example, by piezo effects or electrical pumps. Containers and dispensing devices that can be used in the context of this embodiment are described, for example, in the German unexamined patent application DE 102012222692 A1.

The antiperspirant cosmetic agent can be produced further preferably as a stick, soft solid, cream, gel, roll-on, or loose or pressed powder. The formulation of the antiperspirant cosmetic agents of the invention in a specific delivery form, such as, for example, an antiperspirant roll-on, an antiperspirant stick, or an antiperspirant gel, is based preferably on the requirements of the intended application. Depending on the intended application, the antiperspirant cosmetic agents of the invention can therefore be present in solid, semi-solid, liquid, disperse, emulsified, suspended, gel-like, multiphasic or powder-like form. The term “liquid” in the context of the present invention also covers any type of solid dispersions in liquids. Furthermore, multiphasic antiperspirant cosmetic agents of the invention in the context of the present invention are understood to be agents that have at least 2 different phases with a phase separation and in which the phases can be arranged horizontally, therefore one above another, or vertically, therefore next to one another. The application can occur, for example, with a rollerball applicator or by means of a solid stick.

It can also be preferred in the context of the present invention, if the antiperspirant cosmetic agent is included on and/or in a disposable substrate, selected from the group of wipes, pads, and puffs. Particularly preferred are wet wipes, i.e., preferably individually packaged wet wipes, prefabricated for the user, as they are well known, e.g., from the field of glass cleaning or from the field of moist toilet tissue. Such wet wipes, which advantageously can also include preservatives, are impregnated or treated with an antiperspirant cosmetic agent of the invention and preferably packaged individually. Preferred substrate materials are selected from porous flat cloths. Said cloths include cloths made of woven and nonwoven (fleece), synthetic and natural fibers, felt, paper, or foam, such as hydrophilic polyurethane foam. Deodorizing or antiperspirant substrates preferred according to the invention can be obtained by soaking or impregnation or also by melting-on of an antiperspirant cosmetic agent of the invention onto a substrate.

According to the invention, the antiperspirant cosmetic agent preferably includes at least one other auxiliary substance, selected from the group of (i) emulsifiers and/or surfactants; (ii) thickeners; (iii) chelating agents; (iv) active deodorant substances; (v) mono- and/or polyhydric alcohols and/or polyethylene glycols; (vi) skin-cooling active substances; (vii) pH-adjusting agents; (viii) skin-care active substances, such as moisturizers, skin-soothing substances, skin-lightening substances, and skin-smoothing substances; and (ix) mixtures thereof.

Suitable emulsifiers and surfactants preferred according to the invention are selected from anionic, cationic, nonionic, amphoteric, in particular ampholytic and zwitterionic emulsifiers and surfactants. Surfactants are amphiphilic (bifunctional) compounds, which consist of at least one hydrophobic and at least one hydrophilic moiety. The hydrophobic group is preferably a hydrocarbon group having 8 to 28 carbon atoms, which may be saturated or unsaturated, linear or branched. This C₈-C₂₈ alkyl chain is particularly preferably linear. Emulsifiers and surfactants usable with preference in the context of the present invention are disclosed, for example, in the German unexamined patent applications DE 102012222692 A1, DE 102010063250 A1, and DE 102010055816 A1.

To thicken the antiperspirant cosmetic agents of the invention, substances are used with preference that are selected are from cellulose ethers, xanthan gum, sclerotium gum, succinoglucans, polygalactomannans, pectins, agar, carragheen (carrageenan), tragacanth, gum arabic, karaya gum, tara gum, gellan gum, gelatin, propylene glycol alginate, alginic acids and salts thereof, polyvinylpyrrolidones, polyvinyl alcohols, polyacrylamides, physically (e.g., by pregelatinization) and/or chemically modified starches, acrylic acid-acrylate copolymers, acrylic acid-acrylamide copolymers, acrylic acid-vinylpyrrolidone copolymers, acrylic acid-vinylformamide copolymers, and polyacrylates. Particularly preferred thickeners are selected furthermore from carbomers. Carbomers are thickening crosslinked polymers of acrylic acid, methacrylic acid, and salts thereof. The crosslinking can occur by polyfunctional compounds such as polyalkylene ethers of polysaccharides or polyalcohols, for example, sucrose allyl ethers, pentaerythritol allyl ethers, and propylene allyl ethers. Preferred in the context of the present invention are homopolymers of acrylic acid or salts thereof, which are crosslinked with a pentaerythritol allyl ether, a sucrose allyl ether, or a propylene allyl ether. A thickener usable in the context of the present invention is a copolymer of C₁₀₋₃₀ alkyl acrylate, acrylic acid, methacrylic acid and esters thereof, which is crosslinked with a sucrose allyl ether or a pentaerythritol allyl ether. Carbomer-based thickeners are the products obtainable under the trade name Carbopol® (BF Goodrich, Ohio, USA) such as, for example, Carbopol 934, Carbopol 940, Carbopol 941, Carbopol 971, Carbopol 974, Carbopol EZ2, Carbopol ETD 2001, Carbopol ETD 2020, Carbopol ETD 2050, Carbopol Ultrez 10, Carbopol Ultrez 20, or Carbopol Ultrez 21.

Furthermore, lipophilic thickeners can be used for thickening the antiperspirant cosmetic agents of the invention. Lipophilic thickeners preferred according to the invention are selected from hydrophobized clay minerals, bentonites, pyrogenic silicic acids, and derivatives thereof.

So as to support further the influencing of the sweat gland(s) by the at least one special protein, it can be advantageous to add to the antiperspirant cosmetic agents of the invention at least one chelating agent, in a total amount of 0.01 to 3.0% by weight, preferably of 0.02 to 1.0% by weight, in particular of 0.05 to 0.1% by weight, based on the total weight of the antiperspirant agent of the invention. In the context of the present invention, preferred chelating agents are selected from the group comprising β-alanine diacetic acid, cyclodextrin, diethylenetriamine pentamethylene phosphonic acid, sodium, potassium, calcium disodium, ammonium, and triethanolamine salts of ethylenediaminetetraacetic acid (EDTA), etidronic acid, hydroxyethylethylenediaminetetraacetic acid (HEDTA) and the sodium salts thereof, sodium salts of nitrilotriacetic acid (NTA), diethylenetriaminepentaacetic acid, phytic acid, hydroxypropyl cyclodextrin, methyl cyclodextrin, pentasodium aminotrimethylene phosphonate, pentasodium ethylenediamine tetramethylene phosphonate, pentasodium diethylentriamine pentaacetate, pentasodium triphosphate, potassium EDTMP, sodium EDTMP, sodium dihydroxyethylglycinate, sodium phytate, sodium polydimethylglycinophenol sulfonate, tetrahydroxyethylethylenediamine, tetrahydroxypropylethylenediamine, tetrapotassium etidronate, tetrasodium etidronate, tetrasodium iminodisuccinate, trisodium ethylenediamine disuccinate, tetrasodium-N,N-bis(carboxymethyl)glutamate, tetrasodium-DL-alanine-N,N-diacetate, and desferrioxamine.

The deodorizing action of the antiperspirant cosmetic agents of the invention can be increased further, if at least one active deodorant substance with antibacterial and/or bacteriostatic and/or enzyme-inhibiting and/or odor-neutralizing and/or odor-absorbing action is included in a total amount of 0.0001 to 40% by weight, primarily of 0.2 to 20% by weight, preferably of 1 to 15% by weight, in particular of 1.5 to 5% by weight, based on the total weight of the antiperspirant cosmetic agent of the invention. Provided that ethanol is used in the agents of the invention, in the context of the present invention this is not regarded as an active deodorant substance, but as a component of the carrier. Active deodorant substances preferred according to the invention are disclosed, for example, in the German unexamined patent application DE 102010063250 A1.

Preferred antiperspirant cosmetic agents of the invention include furthermore at least one water-soluble polyhydric C₂₋₉ alkanol with 2 to 6 hydroxyl groups and/or at least one water-soluble polyethylene glycol with 3 to 50 ethylene oxide units and mixtures thereof. These do not include the aforementioned active deodorant substances in the form of 1,2-alkanediols. Preferred alkanols and water-soluble polyethylene glycols are described, for example, in the German unexamined patent application DE 102010063250 A1.

According to another embodiment of the present invention, the antiperspirant cosmetic agents include furthermore at least one skin-cooling active substance. Skin-cooling active substances suitable according to the invention are, for example, menthol, isopulegol, and menthol derivatives, e.g., menthyl lactate, menthyl glycolate, menthyl ethyl oxamates, menthyl pyrrolidone carboxylic acid, menthyl methyl ether, menthoxypropanediol, menthone glycerin acetal (9-methyl-6-(1-methylethyl)-1,4-dioxaspiro(4,5)decane-2-methanol), monomenthyl succinate, 2-hydroxymethyl-3,5,5-trimethylcyclohexanol, and 5-methyl-2-(1-methylethyl)cyclohexyl-N-ethyloxamate. Preferred as skin-cooling active substances are menthol, isopulegol, menthyl lactate, menthoxypropanediol, menthylpyrrolidone carboxylic acid, and 5-methyl-2-(1-methylethyl)cyclohexyl-N-ethyloxamate, and mixtures of said substances, in particular mixtures of menthol and menthyl lactate, menthol, menthol glycolate, and menthyl lactate, menthol and menthoxypropanediol, or menthol and isopulegol.

Used preferably as pH-adjusting agents according to the invention are acids and/or alkalizing agents and/or buffers. Inorganic acids (such as, for example, hydrochloric acid, sulfuric acid, or phosphoric acid) or organic acids (such as, for example, citric acid, tartaric acid, or malic acid) are used with preference as acids according to the invention. The alkalizing agents usable according to the invention are preferably selected from the group, formed by ammonia, basic amino acids, alkali hydroxides, carbonates and hydrogen carbonates, alkanolamines, for example, amino-2-methyl-1-propanol, monoethanolamine, triethanolamine, diethanolamine, and triisopropanolamine, alkali metal metasilicates, urea, morpholine, N-methylglucamine, imidazole, alkali phosphates, and alkali hydrogen phosphates. Lithium, sodium, potassium, in particular sodium or potassium, are preferably used as alkali metal ions. Suitable as buffer systems in the context of the present invention are in particular carbonic acid-bicarbonate buffers, carbonic acid-silicate buffers, acetic acid-acetate buffers, phosphate buffers, ammonia buffers, citric acid or citrate buffers, buffers based on tris(hydroxymethyl)aminomethane, buffers based on 4-(2-hydroxyethyl)-1-piperazineethanesulfonic acid, buffers based on 4-(2-hydroxyethyl)piperazine-1-propanesulfonic acid, buffers based on 2-(N-morpholino)ethanesulfonic acid, and barbital-acetate buffers. The selection of the appropriate buffer system is based in this case on the desired pH of the antiperspirant cosmetic agents of the invention.

The preferred embodiments described below include neither antiperspirant aluminum and/or zirconium halides and/or hydroxyhalides nor bovine serum albumin (BSA):

In a preferred embodiment, the antiperspirant cosmetic agents of the invention are characterized in that, based on the total weight of the antiperspirant cosmetic agent of the invention, they contain
at least one protein in a total amount of 0.5 to 60% by weight, primarily of 1.0 to 50% by weight, preferably of 1.5 to 40% by weight, more preferably of 2.0 to 30% by weight, in particular of 2.0 to 20% by weight,
12 to 98% by weight, preferably 25 to 55% by weight, preferably 30 to 50% by weight, in particular 35 to 45% by weight of water,
at least one emulsifier and/or a surfactant,
at least one pH-adjusting agent,
at least one preservative, and
at least one substance, selected from the group of cosmetic oils that are liquid at 20° C. and 1013 hPa, odorants, and waxes,
   wherein the at least one protein is isolated from human or animal sources or from fish or birds or eggs and wherein the at least one protein causes a change in light absorption of 1 to 100% at a pH value change of at least 0.5 in a pH range of pH 4.0 to pH 8.0, a temperature of 20° C. to 40° C., and a protein concentration of 0.001 to 10% by weight, based on the total weight of the sample mixture used for determining the change in light absorption.

In another preferred embodiment, the antiperspirant cosmetic agents of the invention are characterized in that, based on the total weight of the antiperspirant cosmetic agent of the invention, they contain
at least one protein in a total amount of 0.5 to 60% by weight, primarily of 1.0 to 50% by weight, preferably of 1.5 to 40% by weight, more preferably of 2.0 to 30% by weight, in particular of 2.0 to 20% by weight,
12 to 98% by weight, preferably 25 to 55% by weight, preferably 30 to 50% by weight, in particular 35 to 45% by weight of water,
at least one emulsifier and/or a surfactant,
at least one pH-adjusting agent,
at least one preservative,
0.01 to 2% by weight, preferably 0.1 to 1% by weight, preferably 0.2 to 0.7% by weight, in particular 0.3 to 0.5% by weight of a thickener, and
at least one substance, selected from the group of cosmetic oils that are liquid at 20° C. and 1013 hPa, odorants, and waxes,
   wherein the at least one protein is isolated from human or animal sources or from fish or birds or eggs and wherein the at least one protein causes a change in light absorption of 1 to 100% at a pH value change of at least 0.5 in a pH range of pH 4.0 to pH 8.0, a temperature of 20° C. to 40° C., and a protein concentration of 0.001 to 10% by weight, based on the total weight of the sample mixture used for determining the change in light absorption.

In a preferred embodiment, the antiperspirant cosmetic agents of the invention are characterized in that, based on the total weight of the antiperspirant cosmetic agent of the invention, they contain
at least one protein in a total amount of 0.5 to 60% by weight, primarily of 1.0 to 50% by weight, preferably of 1.5 to 40% by weight, more preferably of 2.0 to 30% by weight, in particular of 2.0 to 20% by weight,
12 to 98% by weight, preferably 25 to 55% by weight, preferably 30 to 50% by weight, in particular 35 to 45% by weight of water,
at least one propellant in a total amount of 1 to 98% by weight, primarily of 20 to 90% by weight, preferably of 30 to 85% by weight, in particular of 40 to 75% by weight,
at least one emulsifier and/or a surfactant,
at least one pH-adjusting agent,
at least one preservative, and
at least one substance, selected from the group of cosmetic oils that are liquid at 20° C. and 1013 hPa, odorants, and waxes,
   wherein the at least one protein is isolated from human or animal sources or from fish or birds or eggs and wherein the at least one protein causes a change in light absorption of 1 to 100% at a pH value change of at least 0.5 in a pH range of pH 4.0 to pH 8.0, a temperature of 20° C. to 40° C., and a protein concentration of 0.001 to 10% by weight, based on the total weight of the sample mixture used for determining the change in light absorption.

In another preferred embodiment, the antiperspirant cosmetic agents of the invention are characterized in that, based on the total weight of the antiperspirant cosmetic agent of the invention, they include
at least one protein in a total amount of 0.5 to 60% by weight, primarily of 1.0 to 50% by weight, preferably of 1.5 to 40% by weight, more preferably of 2.0 to 30% by weight, in particular of 2.0 to 20% by weight,
12 to 98% by weight, preferably 25 to 55% by weight, preferably 30 to 50% by weight, in particular 35 to 45% by weight of water,
at least one propellant in a total amount of 1 to 98% by weight, primarily of 20 to 90% by weight, preferably of 30 to 85% by weight, in particular of 40 to 75% by weight,
at least one emulsifier and/or a surfactant,
at least one pH-adjusting agent,
at least one preservative,
0.01 to 2% by weight, preferably 0.1 to 1% by weight, preferably 0.2 to 0.7% by weight, in particular 0.3 to 0.5% by weight of a thickener, and
at least one substance, selected from the group of cosmetic oils that are liquid at 20° C. and 1013 hPa, odorants, and waxes,
   wherein the at least one protein is isolated from human or animal sources or from fish or birds or eggs and wherein the at least one protein causes a change in light absorption of 1 to 100% at a pH value change of at least 0.5 in a pH range of pH 4.0 to pH 8.0, a temperature of 20° C. to 40° C., and a protein concentration of 0.001 to 10% by weight, based on the total weight of the sample mixture used for determining the change in light absorption.

In the context of the present invention, it can also be provided to use the cosmetic agent of the invention as part of a two-component agent. The individual components for this purpose are preferably stored in separate containers and applied to the skin in any sequence one after another or simultaneously.

A further subject of the present invention therefore is a packaging unit (kit of parts), comprising, packaged separately from one another,
a) at least one first container (C1), containing a cosmetic agent (M1) comprising at least one active antiperspirant substance, and
b) at least one second container (C2), including a cosmetic agent (M2) comprising at least one protein, wherein the at least one protein is isolated from human or animal sources or from fish or birds or eggs, wherein the at least one protein causes a change in light absorption of 1 to 100% at a pH value change of at least 0.5 in a pH range of pH 4.0 to pH 8.0, a temperature of 20° C. to 40° C., and a protein concentration of 0.001 to 10% by weight, based on the total weight of the sample mixture used for determining the change in light absorption, and wherein the cosmetic agent includes no aluminum and/or zirconium halides and/or hydroxyhalides and no bovine serum albumin (BSA).

The term “active antiperspirant substance” according to the invention is understood to mean active substances that prevent or reduce the perspiration of the body's sweat glands. This term does not include, however, the proteins, included in cosmetic agent (M2), from human or animal sources, which cause a change in light absorption under the above-described conditions.

The statements made about the cosmetic agents of the invention apply mutatis mutandis to cosmetic agent (M2) in container (C2).

A further subject of the present invention is the use of a protein for at least partially influencing the sweat gland(s), wherein the at least one protein is isolated from human or animal sources or from fish or birds or eggs, wherein the at least one protein causes a change in light absorption of 1 to 100% at a pH value change of at least 0.5 in a pH range of pH 4.0 to pH 8.0, a temperature of 20° C. to 40° C., and a protein concentration of 0.001 to 10% by weight, based on the total weight of the sample mixture used for determining the change in light absorption, and wherein the at least one protein is not bovine serum albumin (BSA).

Influencing the sweat gland(s) according to the invention is understood to mean influencing the sweat gland(s) with respect to the fact that the secretion of sweat from the excretory duct is prevented or reduced. Without wishing to be restricted to a theory, this can occur, for example, by the formation of a gel and/or depositing of the at least one special protein in the excretory duct of the sweat gland or the excretory ducts of the sweat glands. Furthermore, the use of the at least one special protein, however, can also lead to a disturbance of the charge balance in the excretory ducts of the sweat glands. The statements made about the cosmetic antiperspirant agents of the invention apply mutatis mutandis to the use of the invention.

Moreover, a further subject of the present invention is the use of a combination, including
a) at least one substance, selected from the group of cosmetic oils that are liquid at 20° C. and 1013 hPa, odorants, and waxes,
b) propellant in a total amount of 0 to 99% by weight, based on the total weight of the antiperspirant cosmetic agent, and
c) at least one protein in a total amount of 0.1 to 70% by weight, based on the total weight of the antiperspirant cosmetic agent, wherein the at least one protein is isolated from human or animal sources or from fish or birds or eggs, wherein the at least one protein causes a change in light absorption of 1 to 100% at a pH value change of at least 0.5 in a pH range of pH 4.0 to pH 8.0, a temperature of 20° C. to 40° C., and a protein concentration of 0.001 to 10% by weight, based on the total weight of the sample mixture used for determining the change in light absorption, and
   wherein the combination includes no aluminum and/or zirconium halides and/or hydroxyhalides and no bovine serum albumin (BSA),
   for reducing and/or preventing sweat, in particular axillary sweat, or sweat in other body regions.

The term “combination” in the context of the present invention comprises a mixture of the ingredients a), b), and c) indicated above. The statements made about the antiperspirant cosmetic agents of the invention and the use of the invention apply mutatis mutandis in regard to the use of the aforementioned combination.

Lastly, a further subject of the present invention is a non-therapeutic cosmetic method for preventing and/or reducing body perspiration, in which an antiperspirant cosmetic agent of the invention or a packaging unit of the invention is applied to the skin, in particular to the skin of the armpits, and remains on the skin of the armpits for at least 1 hour, primarily for at least 2 hours, preferably for at least 4 hours, in particular for at least 6 hours.

If in the context of the method of the invention the packaging unit is used according to the invention, it can be provided that cosmetic agent (M1) in container (C1) is applied first and then cosmetic agent (M2) in container (C2). It is also possible, however, that cosmetic agent (M2) in container (C2) is applied first and then cosmetic agent (M1) in container (C1). Moreover, cosmetic agent (M1) in container (C1) and cosmetic agent (M2) in container (C2) can also be applied simultaneously. The time period between the use of both agents (M1) and (M2) is 0 seconds to 24 hours.

The statements made about the antiperspirant cosmetic agents of the invention and the use of the invention apply mutatis mutandis to the method of the invention.

The following examples explain the present invention without however limiting the same:

### Examples

Antiperspirant cosmetic agents of the invention with a pH of 2.5 to 10.0 (quantitative data given in % by weight)

| | 1 | 2 | 3 | 4 | 5 | 6 | 7 |
|---|---|---|---|---|---|---|---|
| Isopropyl | 0.50 | 0.10 | 0.50 | 1.0 | 2.0 | 3.0 | 5.0 |
| myristate | | | | | | | |
| Protein ^{a)} | 0.50 | 2.0 | 3.0 | 5.0 | 7.0 | 10 | 20 |
| Eumulgin B3 ^{b)} | 3.0 | 3.0 | 3.0 | 4.0 | 4.0 | 4.0 | 5.0 |
| Perfume | 0.10 | 0.20 | 0.30 | 0.30 | 0.50 | 0.8 | 1.0 |
| Preservative | 0.50 | 0.50 | 0.50 | 0.80 | 0.80 | 1.5 | 2.0 |
| pH-adjusting | To pH | To pH | To pH | To pH | To pH | To pH | To pH |
| agent | | | | | | | |
| Water | To 100 | To 100 | To 100 | To 100 | To 100 | To 100 | To 100 |

^{a)} Protein from human or animal sources or from fish or birds or eggs
^{b)} Eumulgin B3 (INCI: Ceteareth-30; BASF)

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An antiperspirant cosmetic agent, comprising:
a) at least one substance, selected from the group consisting of cosmetic oils that are liquid at 20° C. and 1013 hPa, odorants, and waxes,
b) a propellant in a total amount of 0 to 99% by weight, based on the total weight of the antiperspirant cosmetic agent, and
c) at least one protein in a total amount of 0.1 to 70% by weight, based on the total weight of the antiperspirant cosmetic agent, wherein the at least one protein is isolated from human or animal sources or from fish or birds or eggs and wherein the at least one protein causes a change in light absorption of 1 to 100% at a pH value change of at least 0.5 in a pH range of pH 4.0 to pH 8.0, a temperature of 20° C. to 40° C., and a protein concentration of 0.001 to 10% by weight, based on the total weight of the sample mixture used for determining the change in light absorption,
wherein the antiperspirant cosmetic agent includes no aluminum and/or zirconium halides and/or hydroxyhalides and no bovine serum albumin (BSA).

2. The antiperspirant cosmetic agent according to claim 1, wherein the at least one protein is included in a total amount of 0.5 to 60% by weight, based on the total weight of the antiperspirant cosmetic agent.

3. The antiperspirant cosmetic agent according to claim 1, wherein the at least one protein is included in a total amount of 2.5 to 30% by weight, based on the total weight of the antiperspirant cosmetic agent.

4. The antiperspirant cosmetic agent according to claim 1, wherein the at least one protein is included in a total amount of 3 to 20% by weight, based on the total weight of the antiperspirant cosmetic agent.

5. The antiperspirant cosmetic agent according to claim 1, wherein the at least one protein has an average molecular weight M_{w} of 150 to 100,000 Da.

6. The antiperspirant cosmetic agent according to claim 1, wherein the at least one protein has an average molecular weight M_{w} of 200 to 10,000 Da.

7. The antiperspirant cosmetic agent according to claim 1, wherein the at least one protein has an average molecular weight M_{w} of 300 to 5000 Da.

8. The antiperspirant cosmetic agent according to claim 1, wherein the at least one protein causes a change in light absorption at a pH change of at least 0.5 in a pH range of pH 4.5 to pH 7.5, at a concentration of 0.001 to 10% by weight protein, based on the total weight of the sample mixture used for the pH measurement, and at a temperature of 20° C.

9. The antiperspirant cosmetic agent according to claim 1, wherein the pH change occurs by adding hydrogen carbonates or carbonates.

10. The antiperspirant cosmetic agent according to claim 1, wherein the pH change occurs by adding sodium hydrogen carbonates.

11. The antiperspirant cosmetic agent according to claim 1, wherein the at least one protein is selected from the group consisting of (i) unmodified proteins; (ii) hydrolyzed proteins; (iii) chemically modified proteins; (iv) physically modified proteins; (v) hydrolyzed unmodified proteins; (vi) hydrolyzed and chemically modified proteins; (vii) hydrolyzed and physically modified proteins; and (viii) mixtures thereof.

12. The antiperspirant cosmetic agent according to claim 11, wherein the protein is a chemically modified protein that is a cationically modified protein that includes one or more groups of the formula R¹—N⁺(CH₃)₂—CH₂—CH(OH)—CH₂—X—R, in which R¹ stands for an alkyl group having 1 to 30 carbon atoms, an alkenyl group having 1 to 30 carbon atoms, a hydroxyalkyl group having 1 to 30 carbon atoms, X for O, N or S, and R for the protein moiety.

13. The antiperspirant cosmetic agent according to claim 1, wherein the at least one protein causes a change in light absorption of 1.5 to 90%.

14. The antiperspirant cosmetic agent according to claim 1, wherein the at least one protein causes a change in light absorption of 3.5 to 60%.

15. The antiperspirant cosmetic agent according claim 1, wherein the concentration of the protein in the mixture, used for determining the change in light absorption, is 0.005 to 10% by weight based on the total weight of the mixture used for determining the change in light absorption.

16. The antiperspirant cosmetic agent according claim 1, wherein the concentration of the protein in the mixture, used for determining the change in light absorption, is 0.09 to 2% by weight by weight based on the total weight of the mixture used for determining the change in light absorption.

17. The antiperspirant cosmetic agent according to claim 1, wherein the at least one protein causes a change in light absorption at a pH change of at least 0.5 and at most 3.5.

18. The antiperspirant cosmetic agent according to claim 1, the antiperspirant cosmetic agent has a pH of 2 to 10.

19. A packaging unit (kit of parts), comprising, packaged separately from one another,
a) at least one first container (C1), containing a cosmetic agent (M1) comprising at least one active antiperspirant substance, and
b) at least one second container (C2), containing a cosmetic agent (M2) comprising at least one protein, wherein the at least one protein is isolated from human or animal sources or from fish or birds or eggs, wherein the at least one protein causes a change in light absorption of 1 to 100% at a pH value change of at least 0.5 in a pH range of pH 4.0 to pH 8.0, a temperature of 20° C. to 40° C., and a protein concentration of 0.001 to 10% by weight, based on the total weight of the sample mixture used for determining the change in light absorption, and wherein the cosmetic agent (M2) includes no aluminum and/or zirconium halides and/or hydroxyhalides and no bovine serum albumin (BSA).

20. A non-therapeutic cosmetic method for preventing and/or reducing body perspiration, in which an antiperspirant cosmetic agent according to claim 1 is applied to the skin, and remains on the skin of the armpits for at least 1 hour.
